(19) United States
(12) Patent Application Publication
(10) Pub. No.: US 2016/0376635 A1
(43) Pub. Date: Dec. 29, 2016

(54) PCR KIT FOR CAPILLARY ELECTROPHORESIS

(71) Applicant: Safran Identity & Security, Issy Les Moulineaux (FR)

(72) Inventors: Marcelin RAGOT, Issy Les Moulineaux (FR); Benoit TONSON, Issy Les Moulineaux (FR); Marina POUET, Issy Les Moulineaux (FR); Rafael ALOS, Issy Les Moulineaux (FR)

(73) Assignees: Safran Identity & Security, Issy Les Moulineaux (FR); Safran Identity & Security, Issy Les Moulineaux (FR)

(21) Appl. No.: 15/190,024

(22) Filed: Jun. 22, 2016

(30) Foreign Application Priority Data
Jun. 23, 2015 (FR) 1555745

(51) Int. Cl. C12Q 1/68; G01N 27/447

(52) U.S. Cl. CPC C12Q 1/686; C12Q 1/6853; G01N 27/44791

(57) ABSTRACT

The present invention relates to a PCR kit for improving detection of particular nucleic acid motifs and for increasing the reliability of the results obtained.

More precisely, the invention proposes to use modified primers having particular characteristics making it possible, during electrophoresis treatment, to detect, on samples containing nucleic acids, the equivalent of a characteristic encoding of particular nucleic acid motifs, this encoding being such that it makes it possible to better detect the presence of these motifs.

The invention also proposes a method for detecting nucleic acid motifs of interest (for example STRs, mini-STRs or VNTRs) using such primers.

It advantageously finds—but is not limited to—application in the field of capillary electrophoresis.

The present invention relates to a PCR kit for improving detection of particular nucleic acid motifs and for increasing the reliability of the results obtained.

More precisely, the invention proposes to use modified primers having particular characteristics making it possible, during electrophoresis treatment, to detect, on samples containing nucleic acids, the equivalent of a characteristic encoding of particular nucleic acid motifs, this encoding being such that it makes it possible to better detect the presence of these motifs.

The invention also proposes a method for detecting nucleic acid motifs of interest (for example STRs, mini-STRs or VNTRs) using such primers.

It advantageously finds—but is not limited to—application in the field of capillary electrophoresis.

### GENERAL TECHNICAL FIELD AND PRIOR ART

Capillary gel electrophoresis is widely used today to obtain DNA profiles which can be used to detect allelic variations and thus to differentiate individuals (Butler, J. M. (1995)).

According to NIST (http://www.cstl.nist.gov/strbase/str_fact.htm), the number of alleles detectable by a conventional PCR kit in the United States (i.e., containing genetic markers obligatory in the United States) is 425 alleles (on the loci: CSF1P0, FGA, TH01, TPDX, VWA, D351358, D5S818, D7S820, D851179, D135317, D165539, D18551, D21511). Moreover, the number of alleles to be detected for a PCR kit commonly used in Europe and able to be used in the United States (i.e., containing obligatory European and American markers) is 552 alleles (on the loci cited above, to which are added the European markers: D151656, D2S441, D251338, D1051248, D125391, D195443, D2251045, SE33).

More precisely, individuals are distinguishable today by the size of short tandem repeat (STR) sequences, which are found in the noncoding portions of many loci. Many multiplex PCR kits which target these STR exist (see for example the following link: http://www.cstl.nist.gov/strbase/multiplx.htm). Traditionally, tested DNA samples undergo PCR and the product of this PCR (i.e., the amplicons generated) is then analyzed by capillary electrophoresis. Each allele contained in the sample contains a precise number of STRs, characteristic of the individual, which influences the size of the amplicon generated by PCR. Each amplicon has a given labeling and size, detectable by a peak on the corresponding electropherogram. The combination of these peaks provides information about the individual's identity.

The electropherogram is the result stemming from capillary electrophoresis, which associates an analog datum read on a CCD sensor with a quantity of material (made detectable by amplification and by labeling using one or more fluorochrome(s)). The larger the value measured by the CCD sensor (up to the saturation limit), the higher the probability that the signal reveals the presence of a nucleic acid of interest, and thus the more reliable the information.

This electropherogram, unfortunately, is prone to diverse variations in quality, mainly due to:
The presence of artifacts generating the appearance of false peaks on the detected signal, these artifacts being able to be due to:
   Polymerase malfunctions. Two phenomena are known:
      One fewer repeat sequence on an STR (known as “polymerase stuttering”);
      Addition of an A nucleotide at the end of copying (known as A addition or shoulder phenomenon on the profiles obtained,
   Digital/analog background noise (related to the CCD sensor, sensor noise, stray light, heat-related optical disturbances, vibrations, low-performance sensor, etc.), which generates a fluctuating signal-to-noise ratio, and
   Saturation phenomena (or “pull-up,” due to faulty calibration of the CCD sensor, faulty color channel separation, a low-performance sensor).
   Fluorochromes remaining free and injected into the capillary (known as a “dye blob”)
   Electric disturbances influencing capillary electrophoresis (known as a “spike”)
The loss of resolution at the end of the electropherogram (due to spreading of the material in the capillary)
The absence of certain peaks due to an amplification (PCR) problem related to primers positioned on a region of mutagenic DNA (phenomenon known as “allele drop-out”

Today, the analog nature of the datum measured by the sensor and the various disruptive parameters coming into play during construction of an electropherogram do not make it possible to avoid a second reading by an expert physical person of the technical field, who must, by virtue of his general knowledge and his experience on the subject, certify the quality of a profile. In general, the expert bases his judgment on the amplitude of the data and the shape thereof (a valid signal has a particular shape, known to the skilled person, because of the phenomenon of loss of resolution throughout the electropherogram, becoming more pronounced toward the end) in order to validate the extraction of a profile. Thus, the current method for analyzing the results cannot be automated, because it is not completely objective in nature. Moreover, it is impossible to assess with what probability the datum selected by the expert is close to reality.

### BRIEF DESCRIPTION OF THE DRAWINGS

FIG. 1 illustrates an exemplary electropherogram which can be obtained with the PCR kit of the invention using a 3-AMC within the meaning of the invention.

FIG. 2 illustrates an exemplary embodiment of the motif method described in the present application, using the PCR kit of the invention with n=3, the motif sought being the combination: T/C, T+1/C and T−1/C−1.

FIG. 3 illustrates a logic diagram representing the technique for detecting a genetic profile according to the present invention.

### GENERAL PRESENTATION OF THE INVENTION

A general aim of the invention is to propose a more reliable and more objective method for detecting the presence of nucleic acid motifs of interest in a sample, from a piece of information read on an electropherogram.

More precisely, the invention proposes a method for reading a nucleic acid profile very robust to signal errors (elimination of stutter, shoulder and pull-up phenomena, etc.), and less sensitive to analog background noise, which ensures a very high objective probability of each piece of information read on an electropherogram. This method avoids a second reading by an expert and can thus be automated.

To that end, the method of the invention requires the use of primer sets specific to the motif sought, hereafter referred to as n-AMC.

These primer sets (n-AMC) are advantageously integrated in a so-called “encoding” PCR kit, which is also an object of the invention. This PCR kit makes it possible to “encode” each motif sought in terms of several variables (base size, color), in a highly specific manner (see examples below). This kit can in particular be used to detect short tandem repeats (STRs), for example STRs having a size of 50 to 450 base pairs (bp). This kit can thus be used in particular in forensic science.

The present invention also relates to a method for detecting motifs of interest in a sample, using such primers or such a kit.

The present invention also relates to a method for reading an electropherogram produced by such a kit.

Finally, the present invention provides recommendations for using these methods, more particularly concerning the parameters of the primers of the invention.

### DESCRIPTION OF THE PRIMER SETS OF THE INVENTION (n-AMC)

Within the meaning of the invention, an “n-AMC” is a set of differentiable primers for specifically amplifying a motif of interest to be detected on a nucleic acid. The number “n” in “n-AMC” indicates the number of primer pairs specific to said motif contained in said set. Thus, a “3-AMC” contains three primer pairs for specifically amplifying the same motif, a “4-AMC” contains four primer pairs for specifically amplifying the same motif, etc.

By “nucleic acid” is meant, within the meaning of the present invention, a nucleic sequence or nucleic acid sequence, a polynucleotide, an oligonucleotide, a polynucleotide sequence or a nucleotide sequence, terms which will be used here indifferently. This term refers to a precise sequencing of nucleotides, comprising (or not) unnatural nucleotides, and corresponds equally well to double-stranded DNA, single-stranded DNA or to transcription products of said DNA. Preferably, it is single-stranded or double-stranded DNA.

As indicated above, each n-AMC contains several primer pairs specific to the same motif of interest (hereafter referred to as “motif A”).

By “motif of interest” is meant a region of a nucleic acid containing a characteristic nucleotide fragment which one seeks to detect and/or to identify. Said fragment can, for example, be a characteristic sequencing of certain bases. Alternatively, said fragment can be a repetition of characteristic sequences, also called repeats (such as short tandem repeats (STRs), mini-STRs or VNTRs), whose number one seeks to measure. Finally, said fragment can have undergone nucleotide insertion or, conversely, nucleotide deletion, relative to a control population. These motifs of interest are preferably the same as those detected by commercial kits (PowerPlex, etc.). In the context of the present invention, said fragment is preferably an STR allele, which, as explained above, can be used to differentiate individuals in forensic science.

By “primers” is meant here a nucleotide fragment comprising, for example, from 15 to 40 nucleotides, in particular from 18 to 30 nucleotides, having a specificity of hybridization under determined conditions for forming a hybridization complex with a target nucleic acid sequence, for example located at the end of a motif of interest as defined above. These primers are preferably single-stranded DNA. To carry out amplification by PCR, it is necessary to use these primers in a “pair,” one primer of the pair hybridizing upstream of the region of interest to be amplified, the other downstream.

By “primer pair specific to motif A” is meant here a primer pair “enabling specific amplification of motif A.” In practice, it is a question of two primers hybridizing specifically on either side of the motif of interest, the two hybridization regions being at most 2000 base pairs apart (Butler M. et al., Fundamentals of Forensic DNA Typing, 2009). By “specifically” is meant that hybridization of said primers does not take place on a nucleic acid having a sequence having less than 80% homology to the region concerned. The parameters defining adequate stringency conditions depend on the temperature at which 50% of the paired strands separate (Tm). For sequences shorter than 30 bases, Tm is defined by the relationship: Tm=4 (G+C)+2 (A+T). Under adequate stringency conditions (i.e., wherein nonspecific sequences do not hybridize), the hybridization temperature is preferably 5 to 10° C. below Tm, and the hybridization buffers used are preferably solutions of high ionic strength (such as 6× SSC solution, for example). The skilled person is quite capable of identifying specific primers for a given nucleotide fragment, once its sequence is known. In forensic science, primer pairs which bind on either side of regions containing STRs, thus making it possible to generate distinct amplicons according to the individuals, are well-known to the skilled person (see the list proposed on the link: http://www.cstl.nist.gov/strbase/primer.htm).

In the context of the invention, it is essential that within each n-AMC at least two primer pairs specific to the same motif generate differentiable amplicons.

By “amplicon” is meant here a nucleotide fragment produced by a polymerization reaction having taken place in the sample tested in the presence of oligonucleotide primers specific to the motif of interest, under suitable polymerization conditions. This amplicon has practically the same nucleotide sequence as the motif of interest (except that its ends consist of the primers used to generate it and possible flanking regions surrounding the region of interest).

By “differentiable” is meant here that it is possible to differentiate each amplicon on the basis of an objective characteristic thereof, detectable by current nucleic acid separation techniques, for example by electrophoresis (on agarose or polyacrylamide gel, possibly capillary) or by chromatography. This characteristic is its size, its labeling, etc.

Thus, each n-AMC contains, for each motif of interest, at least two primer pairs including:
a primer pair specific to motif A, producing an amplicon of size T, detectable during electrophoresis with a color C. This primer pair will hereafter be defined as the “reference” primer pair of the n-AMC for motif A. It is preferably the primer pair commonly used in the kits and methods of the prior art for detecting motif A, and
at least one other primer pair specific to motif A, generating an amplicon differentiable from that produced by the reference pair. This other primer pair is hereafter referred to as the “modified primer” pair.

In a preferred embodiment, amplicons generated by these two primer pairs are characterized by their size (at least one base of difference), or by their labeling, or by their size and their labeling.

In an even more preferred embodiment, the modified primer pair generates an amplicon which contains one base more or fewer relative to that generated by the reference primers, or generates an amplicon which is detectable by a signal different from that carried by the amplicon generated by the reference primers (for example a color C′ distinct from C).

### Kit of the Invention

In a first aspect, the present invention relates to a PCR kit containing, in addition to the reagents commonly used in PCR (dNTPs, Taq polymerase, etc.), an n-AMC as defined above, wherein n is greater than or equal to 2.

More precisely, the present invention relates to a PCR kit containing, for each motif of interest to be identified on a nucleic acid, at least two primer pairs specific to said motif, said pairs generating, for each motif, at least two differentiable amplicons.

In this way, during the implementation of capillary electrophoresis, one has a redundancy in motif detection information (in particular for n=2). Transposed to the digital domain, it is the equivalent of encoding with redundancy and correcting code (in particular for n>2).

According to an embodiment, PCR kit containing, for each motif of interest to be identified on a nucleic acid, at least two primer pairs specific to said motif, said pairs being adapted to generate, for each motif, at least two differentiable amplicons. In a preferred embodiment, said kit contains a 2-AMC, i.e. two primer pairs specific to each motif to be detected, said pairs generating, for each motif to be detected, two differentiable amplicons.

In a preferred embodiment, said kit contains a 3-AMC, i.e. three primer pairs specific to each motif to be detected, said pairs generating, for each motif to be detected, three differentiable amplicons.

Preferably, said amplicons are differentiable by their size, or by their labeling, or by a combination of these characteristics.

More preferably, said primer pairs generate amplicons whose size differs by at least one base and/or generate differentially labeled amplicons.

It is in particular possible to use modified primer pairs generating amplicons whose size differs by one base pair, by two base pairs, by three base pairs, indeed by four base pairs, or by any number of base pairs.

According to an embodiment, the PCR kit contains at least two primer pairs specific to a motif of interest to be identified on a nucleic acid, said pairs being adapted to generate, for said motif, at least two amplicons differentiable by their size and whose size differs by one base pair, by two base pairs, by three base pairs, or by four base pairs.

It is further possible to use modified primer pairs generating differentially labeled amplicons. The “labels” carried by the amplicons can be radioactive isotopes, enzymes (in particular a peroxidase or an alkaline phosphatase), chromophoric chemical compounds, chromogenic, fluorogenic or luminescent compounds, nucleotide-based analogues, or ligands such as biotin or any other equivalent means. Among the radioactive isotopes used, mention may be made of ³²P, ³³P, ³⁵ S, ³H or ¹²⁵I. Nonradioactive entities are selected from ligands such as biotin, avidin, streptavidin, digoxigenin, haptens, dyes, luminescent agents such as radioluminescent, chemiluminescent, bioluminescent, fluorescent or phosphorescent agents. Preferably, the amplicons generated by PCR carry fluorescent labels detectable by capillary electrophoresis such as fluorescein, carboxy-fluorescein, Texas Red, Rhodamine-Red, carboxy-X Rhodamine (CXR), Cyanine, Alexa Fluor dyes, or any other cited on the site http://fr.wikipedia.org/wiki/Fluorochrome. It is in particular possible to use the labels described in Butler M. et al., Fundamentals of Forensic DNA Typing, 2009, in particular 5-FAM (5-carboxy fluorescein), JOE (6-carboxy-2,7-dimethoxy-4,5-dichlorofluorescein), ROX (CXR; 6-carboxy-X-rhodamine), fluorescein, TMR (TAMRA; N,N,N,N-tetramethyl-6-carboxyrhodamine, TET (4,7,2,7-tetrachloro-6-carboxyfluorescein, or HEX (4,7,2,4,5,7-hexachloro-6-carboxyfluorescein).

Such amplicons can be obtained using particular primers, for example primers of different size or carrying a different label.

It is indeed possible to control very precisely the size of an amplicon obtained by PCR by directly modulating the size of the primers used. In particular, it is possible to generate amplicons whose size differs by X base pair(s) using modified primers containing X bases more or fewer relative to the reference primers. More precisely, it is possible to obtain two amplicons having X base pair(s) of difference using two primer pairs identical within about X bases(s) (X bases(s) more or fewer in one primer of the modified primer pair relative to the equivalent primer in the reference primer pair).

In a preferred embodiment, the kit of the invention contains modified primers whose nucleotide sequence is identical to that of the reference primers, within about X bases(s). Preferably, X is an integer between 1 and 10.

It is also possible to generate amplicons of different size using primers whose hybridization region on the nucleic acid of interest is shifted (by X bases) relative to that of the reference primers. Preferably, X is an integer between 1 and 10.

Moreover, it is possible to control very precisely the nature of the label attached to an amplicon using primers which are themselves labeled. More precisely, it is possible to obtain two amplicons differentiable by the label which they carry using two primer pairs carrying a different label (for example a different fluorochrome).

In another preferred embodiment, the kit of the invention thus contains a modified primer pair of which at least one primer carries a label different from the label(s) carried by the reference primers.

In addition to the n-AMC, the PCR kit of the invention can contain any reagent used to carry out PCR, namely a recombinant Taq polymerase, a buffer for keeping reaction medium pH stable (typically Tris-HCl), dNTPs, MgCl₂, etc. The conditions for amplifying a nucleic acid and the reagents to be used for this purpose are well-known to the skilled person. It is possible on this subject to consult molecular biology works, such as Sambrook et al. (Molecular Cloning: A Laboratory Manual, Cold Spring Harbor Laboratory; 3^{rd} edition, 2001).

According to an embodiment, the kit contains a distinct container for each primer of said kit. In this embodiment, each primer is disposed in a distinct container within the kit.

According to another embodiment, the kit contains a distinct container for each primer pair of said kit. In this embodiment, the primers of each primer pair of the kit are premixed two by two in a distinct container for each primer pair.

According to another embodiment, the kit contains a single container for all the primer pairs of said kit. In this embodiment, the primers of each primer pair of the kit are premixed in a single container.

By “container” is meant here a receptacle, a tube, an ampule, a syringe, a cartridge, a bottle, a flask, a vial, a box, a capsule, an envelope, a well, a chamber, a bag, a packet, or a patch. The container can be hermetic or not hermetic, closed for example by a stopper, a capsule, a seal, a film, or not closed.

For n=2, the kit of the invention contains a 2-AMC.

In this particular embodiment, the kit of the invention contains a reference primer pair capable of specifically amplifying motif A, producing an amplicon of size T (T base pairs), labeled with a label M (for example a fluorochrome of color C), and:
a modified primer pair capable of specifically amplifying motif A, producing an amplicon having a size T′ different from T, said amplicon being labeled with a label M (for example a fluorochrome of color C), or
a modified primer pair capable of specifically amplifying motif A, producing an amplicon having a size T, said amplicon being labeled with a label M′ different from M (for example a fluorochrome of color C′ different from C).

Preferably, the size T′ of the amplicon differs from T by one, by two, by three, indeed by four base pairs (more or fewer).

In the more particular case where T′ differs from T by one base pair, the kit of the invention contains:
A reference primer pair capable of specifically amplifying motif A, producing an amplicon of size T (T base pairs), labeled with a label M (for example a fluorochrome of color C), and
A modified primer pair capable of specifically amplifying motif A, producing an amplicon of size T±1 base pairs, labeled with a label M (for example a fluorochrome of color C) or with a label M′ different from M (for example a fluorochrome of color C′ distinct from C).

In this case, for example, it is possible to use a modified primer pair wherein one of the primers contains one base more or fewer relative to the equivalent primer of the reference pair.

Although these parameters do not change the reliability of the kit of the invention, it should be noted that:
Size T−1 by in color C is preferably to be avoided because of shoulder phenomena.
Size T−4 by in color C is preferably to be avoided because of stuttering phenomena.
Size T in the color different from C is preferably to be avoided because of pull-up phenomena.

In addition to this 2-AMC, this PCR kit can contain any reagent used to carry out PCR, namely a recombinant Taq polymerase, a buffer for keeping reaction medium pH stable (typically Tris-HCl), dNTPs, MgCl₂, etc.

For n=3, the kit of the invention contains a 3-AMC.

In another particular embodiment, the kit of the invention contains a 3-AMC. This makes it possible to maximize the number of pieces of information read on the electropherogram while limiting the number of reading errors.

In this case, the kit of the invention contains, for each motif of interest to be identified on a nucleic acid, three primer pairs specific to said motif, said pairs generating three amplicons differentiable by their size and/or their labeling.

In a particular embodiment, the kit of the invention contains a reference primer pair capable of specifically amplifying motif A, producing an amplicon of size T (T base pairs), labeled with a label M (for example a fluorochrome of color C), and at least two primer pairs selected from:
a modified primer pair capable of specifically amplifying motif A, producing an amplicon having a size T′ different from T, said amplicon being labeled with a label M (for example a fluorochrome of color C),
a modified primer pair capable of specifically amplifying motif A, producing an amplicon having a size T, said amplicon being labeled with a label M′ different from M (for example a fluorochrome of color C′ different from C), and
a modified primer pair capable of specifically amplifying motif A, producing an amplicon having a size T′ different from T, said amplicon being labeled with a label M′ different from M (for example a fluorochrome of color C′ different from C).

Preferably, the size T′ of the amplicon differs from T by one, by two, by three, indeed by four base pairs (more or fewer).

In the more particular case where T′ differs from T by one base pair, the kit of the invention contains a reference primer pair capable of specifically amplifying motif A, producing an amplicon of size T (T base pairs), labeled with a label M (for example a fluorochrome of color C), and at least two primer pairs selected from:
a modified primer pair capable of specifically amplifying motif A, producing an amplicon having a size T±1 base pairs, said amplicon being labeled with a label M (for example a fluorochrome of color C),
a modified primer pair capable of specifically amplifying motif A, producing an amplicon having a size T, said amplicon being labeled with a label M′ different from M (for example a fluorochrome of color C′ different from C), and
a modified primer pair capable of specifically amplifying motif A, producing an amplicon having a size T±1 base pairs, said amplicon being labeled with a label M′ different from M (for example a fluorochrome of color C′ different from C).

In particular, the kit of the invention can contain:
A reference primer pair capable of specifically amplifying motif A, producing an amplicon of size T, labeled with a label M (for example a fluorochrome of color C),
A modified primer pair capable of specifically amplifying motif A, producing an amplicon of size T, labeled with a label M′ different from M (for example a fluorochrome of color C′ distinct from C), and
A modified primer pair capable of specifically amplifying motif A, producing an amplicon of size T+1 base pairs, labeled with a label M (for example a fluorochrome of color C).

In another particular embodiment, the kit of the invention can contain:
A reference primer pair capable of specifically amplifying motif A, producing an amplicon of size T, labeled with a label M (for example a fluorochrome of color C),
A modified primer pair capable of specifically amplifying motif A, producing an amplicon of size T+1 base pairs, labeled with a label M (for example a fluorochrome of color C), and
A modified primer pair capable of specifically amplifying motif A, producing an amplicon of size T±1 base pairs, labeled with a label M′ different from M (for example a fluorochrome of color C′ distinct from C).

In addition to this 3-AMC, this PCR kit can contain any reagent used to carry out PCR, namely a recombinant Taq polymerase, a buffer for keeping reaction medium pH stable (typically Tris-HCl), dNTPs, MgCl₂, etc.

### Detecting a Motif of Interest Using the Kit of the Invention

An n-ALC is, within the meaning of the present invention, the visible result of the detection of an n-AMC in a two-dimensional system. More precisely, in the case of capillary electrophoresis, it is thus a set of n peaks detected on the electropherogram, appearing in the neighborhood of the size T expected for the amplicon. Each n-ALC reveals with very high probability the presence (or the absence) of the motif of interest in the nucleic acid sample. This n-ALC makes it possible in particular to characterize objectively which allele(s) is(are) carried by the individual tested.

In a second aspect, the present invention relates to a method for detecting at least one nucleic acid motif of interest in a biological sample, from n-ALCs obtained using the kit of the invention. Naturally, this method makes it necessary to use as many n-AMCs as loci of interest.

This method thus uses the kit of the invention as described above, wherein the number of n-AMCs is adjusted to the number of motifs to be detected.

In an embodiment, the method for detecting at least one nucleic acid motif of interest in a biological sample is characterized in that it uses the kit as defined above and in that said nucleic acid motif of interest is detected when, for said motif, at least one amplicon per specific primer pair of said motif used is detected.

More precisely, the present invention thus comprises the following steps:
a) Obtaining a biological sample containing nucleic acids,
b) Contacting the biological sample with the primer pairs of the n-AMCs described above, and reagents required to amplify said nucleic acids,
c) Amplifying said nucleic acids under appropriate conditions,
d) Detecting the amplicons obtained by means of their size and/or their labeling or a two-dimensional detection system.
e) Generating a genetic profile according to the amplicons detected in step d).

By “biological sample” is meant here any sample likely to contain nucleic acids, in particular a sample containing cells (animal, human, plant, microbial, etc.). Preferably, in the context of application in forensic science, it is a sample of blood, and, more particularly, a sample of serum or plasma taken with no invasive step from an individual (human or animal). It is also possible to use a sample of saliva, of sperm, of hair, or of urine. Finally, it is possible to use contact evidence (skin cells, for example).

In particular cases where the amount of nucleic acids in the sample taken is too small or when said nucleic acids are contained in cells, it is possible to add prior to step b) a step of extraction and/or purification and/or concentration of said nucleic acids. Any technique known to the skilled person for this purpose can thus be used (Chelex extraction, FTA paper, solid-phase extraction using silica columns or magnetic beads.). Such techniques, for example, are described in Sambrook et al. (Molecular Cloning: A Laboratory Manual, Cold Spring Harbor Laboratory; 3rd edition, 2001). In general, the method of the invention can be carried out on a sample containing a small amount of DNA (for example 0.1 to 0.5 ng of DNA). When such steps of purification or concentration are carried out, the biological sample used in step b) is obviously that having been purified and/or concentrated.

To this biological sample is added, during a second step, an adequate number of n-AMCs (as many n-AMCs as motifs to be detected), as well as reagents essential to generating the expected amplicons (a recombinant Taq polymerase, Tris-HCl, dNTPs, MgCl₂, etc.).

The method of the invention contains next a step of amplification c) during which a polymerase chain reaction (PCR) is carried out under conditions usually used by the skilled person (Butler M. et al., Fundamentals of Forensic DNA Typing, 2009). Several alternative techniques to conventional PCR can also be used in this step. These techniques are for example strand displacement amplification (SDA), transcription-based amplification system (TAS), self-sustained sequence replication (3SR), nucleic acid sequence based amplification (NASBA), transcription mediated amplification (TMA), ligase chain reaction (LCR), repair chain reaction (RCR), cycling probe reaction (CPR), or Q-beta-replicase amplification. Generally, it is possible to use in this step any technique resulting in amplification of the target nucleic acid. The skilled person will be able to identify which reagents are required in the reaction medium, in addition to the n-AMCs of the invention, to generate the expected amplicons.

Following this amplification step, the amplicons produced are detected by means of a two-dimensional detection system (size/color, for example). Preferably, this detection system is electrophoresis or chromatography. More preferably, the detection system is capillary electrophoresis.

Finally, the signals corresponding to the labeled amplicons are analyzed in order to decide on the presence or the absence of the motifs of interest in the sample.

This analysis step is preferably done automatically, for example using software capable of recognizing the signals (or the sets of n peaks or the n-ALCs) and attributing them to a phenotype (according to one of the two methods of analysis described below).

The present invention proposes two different methods for analyzing the signals/sets of n peaks/n-ALCs obtained by the method of the invention and for deciding on the presence of the motif of interest accordingly.

First Method of Analysis (so-called “Dictionary” Method):

This first method of analysis requires constructing or obtaining, prior to the analysis, a “dictionary” or “catalog” listing, for each motif of interest, all the n-ALCs that it is theoretically possible to detect using the n-AMCs used.

In other words, it is advisable to compile—or to acquire—the list of all the signal combinations expected for each n-AMC selected. This list of n-ALCs constitutes the dictionary of valid alleles. It is, for example, created from the lists typically provided in commercial kits (PowerPlex, etc.). These combinations are easily identifiable, for each motif of interest, once the reference primers and the modified primers have been chosen, since this choice determines the size of the amplicon expected in the presence of the motif of interest versus in its absence, as well as the labeling expected for each type of amplicon.

For example, to detect the 425 possible alleles required by American regulations, said dictionary will list 425 n-ALCs. Moreover, to detect the 552 possible alleles required by European and American regulations, said dictionary will list 552 n-ALCs.

To obtain the list of motifs truly present in the sample, all the combinations of n peaks detected for each n-ALC of the sample are then compared with the n-ALCs expected for these motifs (i.e., those compiled beforehand in the list). This list of n-ALCs constitutes the dictionary of valid alleles.

The probability of obtaining a valid n-ALC by this method is 1−X/Cⁿ_{R}, where X is the number of motifs to be detected, R is the number of different sizes detectable (all colors combined) and n is the number of primer pairs used for each motif.

Insofar as each color channel can produce as many peaks as possible chain sizes (in bp), it is possible to calculate that, in a conventional system containing five color channels (such as those proposed by GlobalFiler), the number of peaks which can be detected on the electropherogram is 1850.

Thus, for such a system, R=1850 and it will thus be possible to generate Cⁿ₁₈₅₀ n-ALCs.

All having an identical probability of existing on the electropherogram as an analog signal, the probability that an n-ALC is valid/expected is thus 1-425/Cⁿ₁₈₅₀ (American system) or 1-552/Cⁿ₁₈₅₀ (American and European systems). If 3-AMCs are used, the probability that a combination detected is a valid n-ALC is 1−(425/C³₁₈₅₀)=1−4.034×10⁻⁷ or 99.99996% (American system) or 1-(552/C³₁₈₅₀) or 99.99995% (American and European systems).

With this “dictionary”-based method, each valid combination identified makes it possible to conclude with certainty that the motif of interest was present in the sample tested. To summarize, it is proposed here a method wherein, to detect said nucleic motif of interest, all the combinations of n peaks detected in the sample for each n-ALC are compared with the expected n-ALCs contained in a list compiled beforehand.

In a preferred embodiment, the kit of the invention contains, in addition to the elements defined above, a document describing all of the n-ALCs expected in a sample (the “catalog” or “dictionary” as described above), which will make it possible to identify to which motifs the detected n-ALCs correspond.

### Second Method of Analysis (So-Called “Moti” Method):

The first method of analysis, described above, makes it possible to detect the presence of a motif of interest whose structure is expected (for example, a given number of repeats within an STR, which had been listed in the “dictionary”). However, insofar as the “dictionary” cannot be exhaustive as for all motifs existing in nature, situations can exist where the motif detected does not correspond to any of those listed.

In particular, this first method does not make it possible to identify the existence of motifs which would be specific to an individual, and, as a result, not listed in the “dictionary” (a rare microvariant, for example).

Detecting these rare motifs is problematic with the methods currently used in forensic science. Indeed, it is sometimes difficult to know if they are true microvariants or if the signal detected corresponds in fact to a peak having migrated poorly.

To improve this point, the invention proposes a second method for analyzing n-ALCs obtained by the method of the invention using n-AMCs, with a view in particular to detecting the presence of rare motifs within a sample.

This second method is based on the fact that each set of n peaks forms a specific n-ALC. Each valid n-ALC detected thus corresponds to the motif of interest sought (for example, for a 3-AMC as described above, an n-ALC can be: size T on color C, size T+1 on color C and size T−1 on color C−1).

Generally, the probability of obtaining a valid n-ALC with this “motif” method is 1−((1/R)*(1/R-1)*( . . . )*(1/R−(n−1))), R being the number of different sizes detectable (all colors combined) and n being the number of primer pairs used for each motif.

For a system containing 1850 possible peaks on the electropherogram, the probability that a detected combination corresponds to a valid n-ALC is 1-(1*(1/1849)*(1/1848))=99.99997%.

Thus, with this second method, the presence of a motif of interest can be identified with certainty. It is also possible to measure the size of the motif of interest.

To summarize, it is proposed here a method wherein, to detect said nucleic motif of interest, the following steps are implemented:
a) detecting all valid n-ALCs in the sample tested,
b) attributing each valid n-ALC to a motif of interest sought.

The use of either of these two methods of analysis has the advantage that peaks associated with artifacts will not form valid n-ALCs, and thus cannot be confused with the motif of interest. Thus, each valid n-ALC detected (i.e., each signal combination generated by an n-AMC) makes it possible to conclude that the motif of interest is present in the sample (“dictionary” method) or that a variant of this motif is present in the sample (“motif” method).

It should be noted that the two methods of analysis can be combined and carried out successively or simultaneously.

Moreover, they can be carried out automatically.

More generally, the method of the invention is characterized in that the steps of detecting and generating a genetic profile, d) and e), and if need be the steps of amplifying and contacting, are carried out automatically.

In a preferred embodiment, the kit of the invention contains n-AMCs with n>2. Indeed, in this case, each n-ALC has a portion of code acting as an “error correcting code” for detecting the motif of interest even if it is little differentiable from noise.

When n>2, it is indeed possible to create n-ALCs containing a unique combination of n−1 elements (“n−1ALCs”), which can be found in the list of expected n-ALCs.

With the “dictionary” method of analysis, it thus suffices to have n−1 elements of an n-ALC in order to identify an n-ALC (and thus the corresponding motif of interest). The probability of having a valid piece of information is 1−(X/Cⁿ⁻¹_{R}), where X is the number of motifs of interest to be detected, R is the number of different sizes detectable (all colors combined) and n is the number of primer pairs used for each motif. For a system containing 1850 possible peaks on the electropherogram, the probability that a corrected combination (via a correcting code) is valid is, for the American system, 1−(425/C²₁₈₅₀)=1−2.485×10⁻⁴=99.9752%, and, for the American and European systems, 1−(552/C²₁₈₅₀)=99.9677%.

With the “motif” method of analysis, the probability of having a valid piece of information is 1−((1/R)*(1/R−1)*( . . . )*(1/R−(n−2))) where R is the number of different sizes detectable (all colors combined) and n is the number of primer pairs used for each motif. For a system containing 1850 possible peaks on the electropherogram, the probability that a corrected combination (via a correcting code) is valid is 1-(1*(1/1849)), or 99.946%.

During analysis of the signals obtained by the method of the invention, signal-to-noise separation is typically carried out by 10 σ thresholding and peak shape detection. However, these methods do not make it possible to produce an objective probability of the reality of the information read. 10 σ thresholding could produce one, but the noise distribution does not follow a known law (normal distribution) on an electropherogram. Moreover, peak shape detection is based on descriptions and empirical observations of the shape that a peak should have.

In the context of the present invention, considering the low probability of obtaining a false n-ALC, it is possible to make a selection of analyzed signals much below what is actually made (10 σ thresholding and peak shape detection). In particular, 3 σ thresholding suffices, in the context of the methods of the invention, for separating signal from noise while producing more information than current methods. Indeed, 3 σ rather than 10 σ thresholding provides a substantial acceptance of noise relative to the probability that an n-ALC is valid.

Concerning 3σ thresholding, it is preferable, before reading the n-ALCs, to use an echo cancellation filter on the electropherogram signal. The echo sought corresponds to stutter and shoulder phenomena. Echo cancellation methods are known to the skilled person relating to signal processing (telephony, network communications, etc.). The methods to be used are the simplest: the echo parameters are fixed: echo at −1 by for shoulder and echo at −4 by for stutter. It is also possible to seek frequency variations in the n-ALCs rather than peaks (noise has a relatively constant frequency, decreasing with nucleic acid length). This is particularly preferable when the polynucleotides detected are large.

The search for peaks and frequency variations can be carried out in combination.

During analysis of the signals obtained by the method of the invention, cases exist for which portions of n-ALCs are covered (completely or partially) by other n-ALCs. This is not a problem, since the method of the invention requires taking into account all the combinations/motifs of n peaks found. Only valid n-ALCs will be retained with the associated probabilities indicated above.

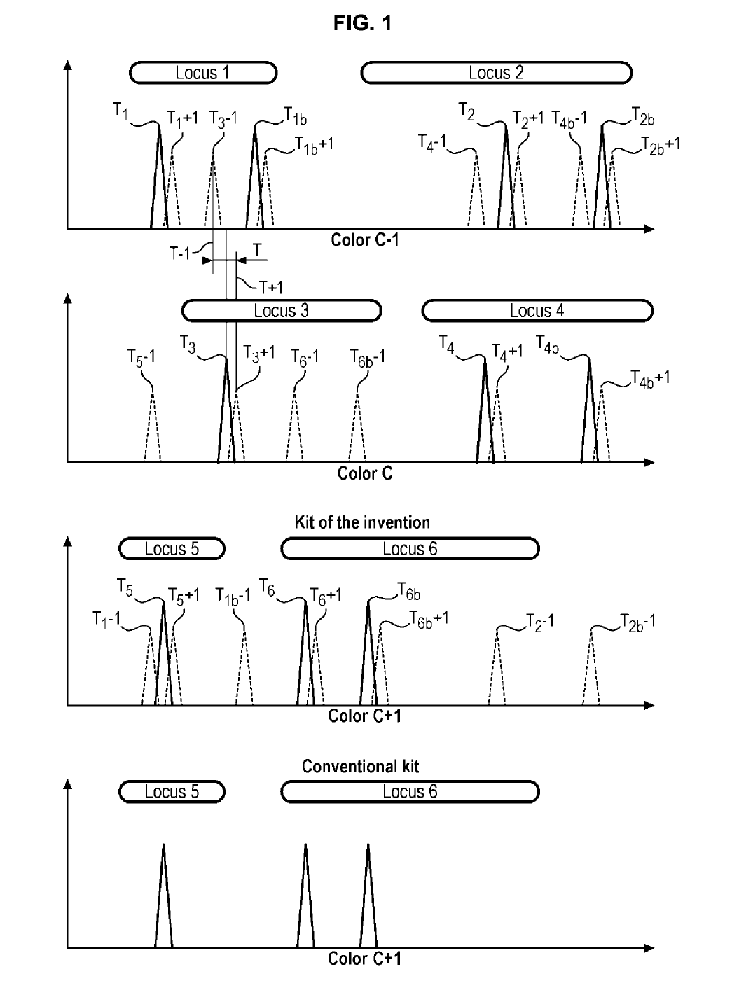

FIG. 1 illustrates an exemplary electropherogram which can be obtained with the PCR kit of the invention using a 3-AMC within the meaning of the invention (three primers for detecting each allele of the locus of interest), using three colors (C−1, C and C+1). The solid lines represent peaks obtained conventionally with commercial kits (see also the electropherogram obtained with a “conventional” kit, the last panel at the bottom of the figure). The solid and dotted lines represent peaks obtained using the PCR kit of the invention (Tₓ=peak corresponding to an amplicon of size T for the allele of locus X, T_{xb}=peak corresponding to the amplicon of the second allele of locus X if the individual is heterozygous, Tₓ+1=peak corresponding to the amplicon of size T+1 base pairs for the allele of locus X, Tₓ−1=peak corresponding to the amplicon of size T−1 base pairs for the allele of locus X). X is here an integer between 1 and 6.

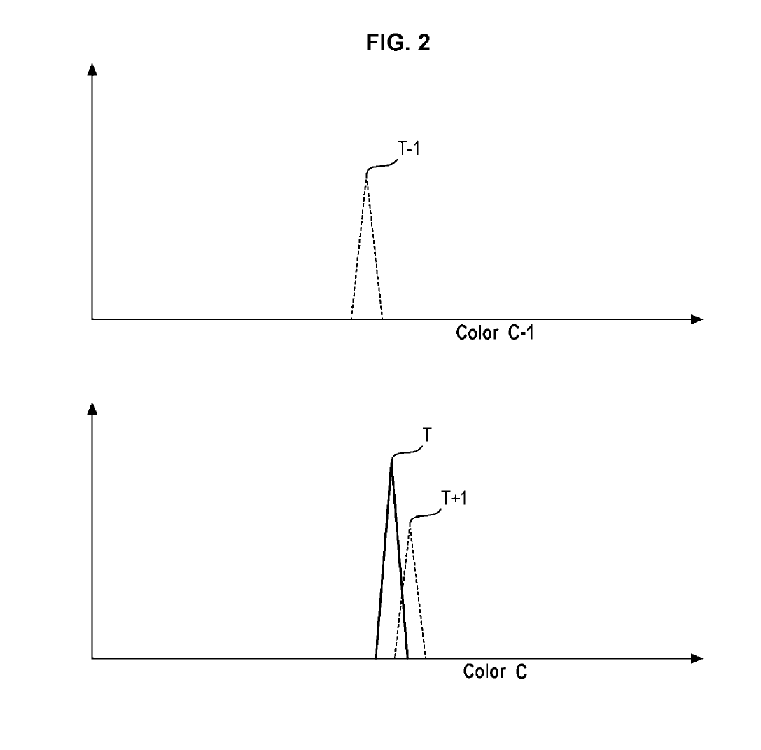

FIG. 2 illustrates an exemplary embodiment of the motif method described in the present application, using the PCR kit of the invention with n=3, the motif sought being the combination: T/C, T+1/C and T−1/C−1. The two graphs represent the electropherograms obtained for colors C−1 (at top) and C (at bottom). The solid line represents the peak obtained conventionally with a commercial kit (corresponding to an amplicon of size T), the dotted lines represent those obtained with the labels of the invention (generating amplicons of size T−1 and T+1 on colors C−1 and C, respectively).

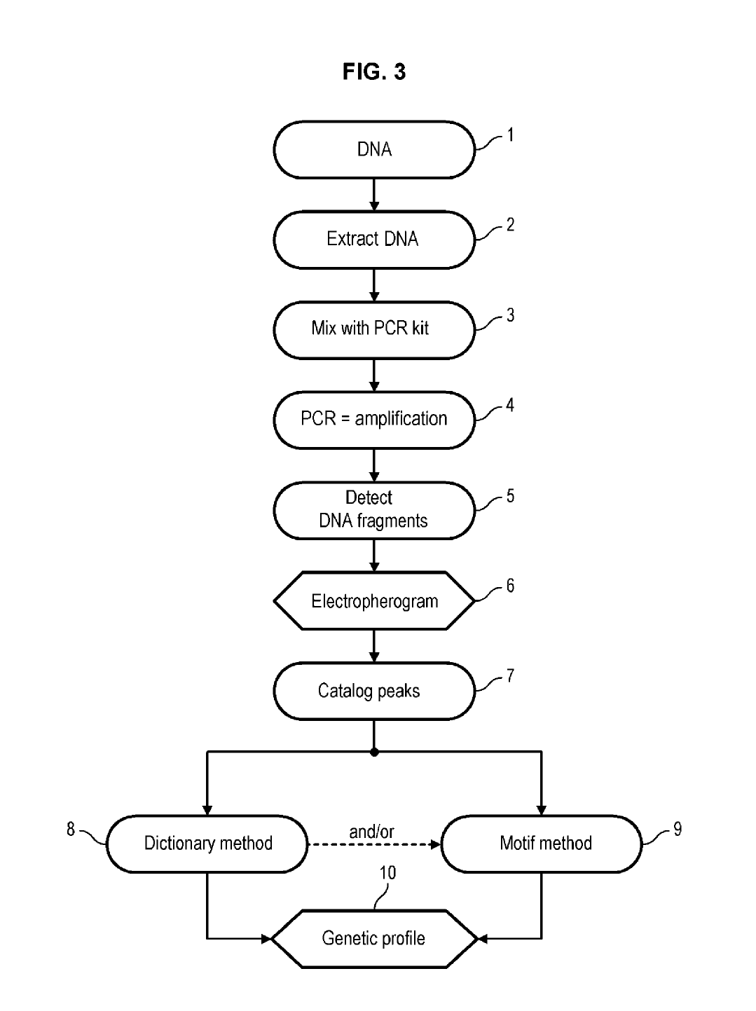

FIG. 3 illustrates a logic diagram representing the technique for detecting a genetic profile according to the present invention. From an individual's sample containing DNA (1), DNA is extracted with conventional techniques (2) then contacted with the components (dNTPs, primers, polymerase, etc.) of the PCR kit of the invention (3). A PCR is carried out (4) then the sample containing the amplicons is analyzed by capillary electrophoresis (5), generating an electropherogram (6) on which the peaks corresponding to the labeled amplicons are compiled (7). The analysis of these peaks can be carried out either by the dictionary method (8) or by the motif method (9), indeed by both methods in combination, so as to detect the individual's genetic profile (10). This detection is more reliable than current techniques and can be done automatically.

1. PCR kit containing at least two primer pairs specific to a motif of interest to be identified on a nucleic acid, said pairs being adapted to generate, for said motif, at least two amplicons differentiable by their size and whose size differs by one base pair, by two base pairs, by three base pairs, or by four base pairs.

2. PCR kit according to claim 1, wherein said primer pairs are adapted to generate, for each motif, amplicons carrying a different label.

3. Kit according to claim 1, containing:
a reference primer pair adapted to specifically amplify motif A, producing an amplicon of size T (T base pairs), labeled with a label M, and
a modified primer pair adapted to specifically amplify motif A, producing an amplicon of size T′ different from T by one, by two, by three or by four base pairs more or fewer, labeled with a label M′ different from M.

4. Kit according to claim 1, containing, for each motif of interest to be identified on a nucleic acid, three primer pairs specific to said motif, said pairs being adapted to generate at least three amplicons differentiable by their size and/or their labeling.

5. Kit according to claim 1, containing a reference primer pair capable of specifically amplifying motif A, producing an amplicon of size T (T base pairs), labeled with a label M, and at least two other primer pairs selected from:
a modified primer pair adapted to specifically amplify motif A, producing an amplicon having a size T′ different from T by one, by two, by three or by four base pairs more or fewer, said amplicon being labeled with a label M,
a modified primer pair adapted to specifically amplify motif A, producing an amplicon having a size T, said amplicon being labeled with a label M′ different from M, and
a modified primer pair adapted to specifically amplify motif A, producing an amplicon having a size T′ different from T by one, by two, by three or by four base pairs more or fewer, said amplicon being labeled with a label M′ different from M.

6. Method for detecting at least one nucleic acid motif of interest in a biological sample, wherein it uses a kit according to claim 1 and in that said nucleic acid motif of interest is detected when, for said motif, at least one amplicon per primer pair used specific of said motif is detected.

7. Method according to claim 6, comprising the following steps:
a) Obtaining a biological sample containing nucleic acids,
b) Contacting the biological sample with the at least two primer pairs and reagents required to amplify said nucleic acids,
c) Amplifying said nucleic acids under suitable conditions,
d) Detecting the amplicons obtained by means of their size and/or their labeling,
e) Generating a genetic profile according to the amplicons detected in step d).

8. Method according to claim 6, wherein said amplicons are detected by means of capillary electrophoresis.

9. Method according to claim 6, wherein said motif of interest is an STR.

10. Method according to claim 6, wherein, to detect said nucleic motif of interest, all the combinations of n peaks detected in the sample for each set of n peaks obtained are compared with the expected sets of n peaks contained in a list compiled beforehand.

11. Method according to claim 6, wherein, to detect said nucleic motif of interest, the following steps are carried out:
a) detecting all the valid sets of n peaks in the sample tested,
b) attributing each valid set of n peaks to a motif of interest sought.

12. Method according to claim 7 wherein the steps of detecting and generating a genetic profile, d) and e), and if need be the steps of amplifying and contacting, are carried out automatically.
